(19) United States
(12) Patent Application Publication
(10) Pub. No.: US 2008/0031914 A1
(43) Pub. Date: Feb. 7, 2008

(54) Flowable biomaterial composition

(76) Inventors: Susan J. Drapeau, Cordova, TN (US); Kathy L. Chamness, Memphis, TN (US)

Correspondence Address: FOX ROTHSCHILD, LLP, 997 LENOX DRIVE, LAWRENCEVILLE, NJ, 08648, US

(21) Appl. No.: 11/497,837

(22) Filed: Aug. 2, 2006

(51) Int. Cl. A61F 2/02; A61K 33/42

(52) U.S. Cl. 424/423; 424/602

(57) ABSTRACT

A putty form biomaterial composition made of a collagen scaffold and ceramic granule having improved flowable yet cohesive characteristics through the addition of, either individually or in some combination, polyhydroxy compounds, a liquid polyhydroxy compound ester, a liquid solution of solid polyhydroxy compound, a liquid solution of solid polyhydroxy compound ester to allow for use in surgical bone repair is presented. Specific polyhydroxy compounds, including polyethylene glycol polymers (PEG), PPO/PEO block co-polymers (i.e., a poloxamer NF grade), and the polysaccharides alginate and chitosan may be utilized.

### FIELD OF THE INVENTION

The present invention relates to a flowable yet cohesive biomaterial composition, and more particularly, to an improved collagen/ceramic putty provided for use in surgical bone repair.

### BACKGROUND OF THE INVENTION

The human skeleton is made up of 206 bones. The bones, also called osseous tissue, are a type of hard endoskeletal connective tissue that support body structures, protect internal organs, and (in conjunction with muscles) facilitate movement. Unfortunately, bones are subject to becoming cracked, splintered, or bisected as a result of physical trauma. Additionally, a bone fracture can also occur as a result of certain medical conditions that weaken the bones, such as osteoporosis or certain types of cancer. Lastly, throughout a person's life, bones are subject to a process, called remodeling, of resorption followed by replacement of bone with little change in shape.

Fractured bones heal by natural processes that start when the injured bone and surrounding tissues bleed. The blood coagulates to form a blood clot situated between the broken fragments. Within a few days blood vessels grow into the jelly-like matrix of the blood clot. The new blood vessels bring white blood cells to the area, which gradually remove the non-viable material. The blood vessels also bring fibroblasts in the walls of the vessels and these multiply and produce collagen fibers. In this way the blood clot is replaced by a matrix of collagen. Collagen's rubbery consistency allows bone fragments to move only a small amount unless severe or persistent force is applied.

At this stage, some of the fibroblasts begin to lay down bone matrix (calcium hydroxy-apatite) in the form of insoluble crystals. This mineralization of the collagen matrix stiffens it and transforms it into bone. In fact, bone is a mineralized collagen matrix; if the mineral is dissolved out of bone, it becomes rubbery. Healing bone callus is on average sufficiently mineralized to show up on X-ray within 6 weeks in adults and less in children. This initial “woven” bone does not have the strong mechanical properties of mature bone. By the process of remodeling, the woven bone is replaced by mature “lamellar” bone. The whole process can take up to 18 months, but in adults the strength of the healing bone is usually 80% of normal by 3 months after the injury.

Additionally, although the process of bone healing is natural, untended and unsupported fractures can lead to mis-grown bone. Therefore, bone fractures are typically treated by restoring the fractured pieces of bone to their natural positions (if necessary), and maintaining those positions while the bone heals. To this end, a fractured limb is usually immobilized with a plaster or fiberglass cast which holds the bones in position and immobilizes the joints above and below the fracture.

Sometimes bones are reinforced with metal, but these fracture implants must be designed and installed with care. For instance, surgical nails, screws, plates and wires are some of the metal pieces used to hold the fractured bone together more directly. However, a problem arises, referred to as stress shielding, when plates or screws carry too large of a portion of the bone's load, causing the bone to atrophy. This problem is reduced, but not eliminated, by the use of low-modulus materials, including titanium and its alloys. The heat generated by the friction of installing hardware can easily accumulate and damage bone tissue, reducing the strength of the connections. If dissimilar metals are installed in contact with one another (i.e., a titanium plate with cobalt-chromium alloy or stainless steel screws), galvanic corrosion will result. The metal ions produced can damage the bone locally and may cause systemic effects as well.

Thus, as can be seen, to both increase the speed by which woven bone is replaced and to prevent damage to bone from the use of metals, a collagen/mineral implant is needed.

### SUMMARY OF THE INVENTION

Accordingly, the present invention introduces a collagen/ceramic putty, having greater cohesive and flowable characteristics, which is designed to allow surgeons to have a malleable implant that localizes biological components and allows a bone graft to be shaped based on the surgical environment and patient anatomy.

The biomaterial composition is a malleable/cohesive, osteo-conductive scaffold composed of collagen that is physically mixed with resorbable ceramic granules. The collagen/ceramic putty can be combined with either bone marrow aspirate or sterile water and then gently packed into voids or gaps of the skeletal system.

The biomaterial composition includes a granular biphasic calcium phosphate (BCP) dispersed within a malleable, aqueous collagen carrier. In addition to the biocompatible carrier, to improve the flowability and/or cohesiveness of the biomaterial composition, a member of the group consisting of liquid polyhydroxy compound, liquid polyhydroxy compound derivative, liquid solution of solid polyhydroxy compound, liquid solution of solid polyhydroxy compound derivative and mixtures thereof may be added.

The present invention, including its features and advantages, will become more apparent from the following detailed description.

### DETAILED DESCRIPTION OF THE INVENTION

The present invention is directed towards an improved putty form biomaterial composition made up from a combination of a medical grade purified collagen and a biphasic calcium phosphate (BCP) ceramic granules for use in packing into bony voids or gaps in the skeletal system created by osseous defects or osseous defects created from traumatic injury to the bone.

The collagen component of the putty form biomaterial composition is a Type 1 bovine collagen. The highly purified resorbable bovine Type 1 collagen is composed of two formulations of collagen, that is, a 70% insoluble fibrous collagen and a 30% soluble collagen. This allows the material to be malleable, non-water soluble, and maintain graft integrity. Preferably, the collagen in the carrier is a mixture of Semed F (insoluble collagen fibers) and Semed S (acid-soluble collagen) that are prepared from bovine hides, and contain telopeptides. Thus, the dry weight ratio of the collagen content is 70% of insoluble collagen fibers (Semed F collagen) to 30% acid-soluble collagen (Semed S collagen), and preferably contains 10.5% to 17% nitrogen and 10.5% to 14% hydroxyproline (average percentage by mass of the collagen portion).

The biphasic ceramic portion is provided in a 15% hydroxyapatite (HA) and 85% beta-tri-calcium phosphate (TCP) formulation. The 15% HA is uniformly distributed through the 85% beta-TCP. HA is a slow resorbing mineral that allows time for the remodeling process to occur, while the beta-TCP is a quicker resorbing mineral. The resorbable ceramic granules are thus optimized to balance bony in-growth and resorbtion of the scaffold structure. The physical structure emulates the highly osteo conductive porous structure of the human cancellous bone, allowing for long-term stability and complete resorption. The composition is thus 80% porous versus 55 to 90% for human cancellous bone. Preferably the average pore size is on average 500 microns which is equivalent to the 500 microns for human cancellous bone. The granules are preferably 0.5 to 1.6 millimeters in diameter, and contain an 80% mineral content (average percentage by mass of 3 measurements, with a tolerance of plus or minus 3 percent).

Bone marrow aspirate (BMA), sterile water or other suitable hydrating agents may be added to the biomaterial composition prior to implantation. Such other hydrating agents include for example, blood, saline or other fluids designed to allow the material to set up in situ. Preferably, the ratio of BMA and/or sterile water to the putty form biomaterial composition is in a 1:1 ratio (e.g., 1 ml of sterile water for each 1 cc of putty). Functionally, the carrier component of the biomaterial composition serves to provide a flowable material of widely varying consistency. The term “flowable” in this context applies to compositions whose consistencies range from those which can be described as shape-sustaining but readily deformable, e.g., those which behave like putty, to those which are runny. Specific forms of flowable biomaterial compositions include cakes, pastes, creams and fillers.

In addition, for improved flow and cohesive characteristics, a structural polysaccharide or, either individually or in some combination, polyhydroxy compounds, a liquid polyhydroxy compound ester, a liquid solution of solid polyhydroxy compound, a liquid solution of solid polyhydroxy compound ester, can be added.

The expressions “liquid polyhydroxy compound” and “liquid polyhydroxy compound derivative” as employed herein are intended to include those compounds of this type which in the pure or highly concentrated state and at ambient temperature, e.g., 15°-40° C., are flowable liquids. The expressions “solid polyhydroxy compound” and “solid polyhydroxy compound derivative” as employed herein are intended to include those compounds of this type which in the pure or concentrated state and at ambient temperature are normally solid or semi-solid but are soluble in a suitable solvent, e.g., water, physiological saline, ethanol, glycerol, glucose, propylene glycol, polyethylene glycol of from 200-1000 molecular weight, etc., or mixtures thereof, to provide a liquid composition.

Useful polyhydroxy compounds possess from 2 to about 18 carbons and include such classes of compounds as the acyclic polyhydric alcohols, non-reducing sugars, sugar alcohols, sugar acids, monosaccharides, disaccharides, water-soluble or water dispersible oligosaccharides, polysaccharides and known derivatives of the foregoing. Specific polyhydroxy compounds include ethylene glycol, diethylene glycol, triethylene glycol, 1,2-propanediol, trimethylolethane, trimethylopropane, erythritol, pentaerythritol, polyalkylene glycols such as the polyethylene glycols, xylitol, sorbitol, mannitol, dulcitol, arabinose, xylose, ribose, adonitol, arabitol, rhamose, inositol, fructose, galactose, glucose, mannose, sorbose, sucrose, maltose, lactose, maltitol, lactitol, stachyose, maltopentaose, cyclomaltohexaose, carrageenan, agar, alginic acid, guar gum, gum tragacanth, locust bean gum, gum arabic, xanthan gum, amylose, mixtures of any of the foregoing, and the like.

Derivatives of the foregoing polyhydroxy compounds, in particular, ester derivatives thereof, are also useful. For example, liquid and solid monoesters and diesters of glycerol can be used to good effect, the solid esters being dissolved up to the limit of their solubilities in a suitable vehicle, e.g., propylene glycol, glycerol, polyethylene glycol of 200-1000 molecular weight, etc. Liquid glycerol esters include monacetin and diacetin and solid glycerol esters include such fatty acid monoesters of glycerol as glycerol monolaurate which is preferred, glyceryl monopalmitate, glyceryl monostearate, etc. An especially preferred carrier herein comprises glyceryl monolaurate dissolved in glycerol or a 4:1 to 1:4 mixture of glycerol and propylene glycol.

Of the foregoing polyhydroxy compounds, glycerol and its liquid monoesters and diesters, e.g., monacetin and diacetin, fructose, glucose and sucrose, and mixtures thereof are preferred. Where the polyhydroxy compound is a solid, e.g., sucrose, a solvent such as water, glycerol, polyethylene glycol of from 200-1000 average molecular weight, or mixture thereof, is used to provide a flowable solution or paste of the compound.

In addition to the specific polyhydroxy compounds mentioned above, also included can be polyethylene glycol polymers (PEG), PPO/PEO block co-polymers (i.e., a poloxamer NF grade) and the polysaccharides alginate and chitosan may be utilized. A PEG may be used, as although it is a water-soluable, waxy solid, its solubility is greatly reduced at low temperatiures (e.g., 0° C.). A poloxamer block copolymer, that is made up of a synthetic copolymer of ethylene oxide and propylene oxide, can be used where a solubilizer and stablilizer that conforms to the NF 19 specifications is needed. Alginate, produced by brown seaweeds, may be used where a thermally stable cold setting in the presence of ions is needed. Chitosan, a linear polysaccharide produced from chitin (the structural element in the exoskeleton of crustaceans), may be used where a substance that is positively charged—and soluble in acidic to neutral solution is needed (e.g., in a negatively charged surface such as a mucosal membrane).

In addition, biomaterial compositions for use in the present invention may further include one or more biocompatible fluid lubricant, such as, for example, hyaluronic acid, dextran sulfate, dextran, succinylated noncrosslinked collagen, methylated noncrosslinked collagen, glycogen, glycerol, dextrose, maltose, triglycerides of fatty acids (such as corn oil, soybean oil, and sesame oil), and egg yolk phospholipid.

Various particulate materials may also be incorporated into biomaterial compositions for use in the invention. Suitable particulate materials include, without limitation, ceramic particles; particulate crosslinked or non-crosslinked fibrillar collagen; poly(lactic) acid (PLA), poly(glycolic) acid (PGA), and copolymers thereof (PLGA); calcium carbonate; calcium sulfate; gelatin beads; polytetrafluoroethylene beads; silicone rubber beads; beads of various hydrogel polymers (such as polyacrylonitrile-polyacrylamide hydrogels); silicon carbide beads; and glass beads.

Biomaterial compositions for use in the invention may also incorporate one or more biologically active agent. The term “biologically active agent” or “active agent” as used herein refers to organic molecules which exert biological effects in vivo. Examples of active agents include, without limitation, enzymes, receptor antagonists or agonists, hormones, growth factors, autogenous bone marrow, antibiotics, antimicrobial agents, and antibodies. The term “active agent” is also intended to encompass various cell types which can be incorporated into the compositions of the invention. The term “active agent” is also intended to encompass combinations or mixtures of two or more active agents, as defined above.

Preferred active agents for use in methods of the present invention include growth factors, such as transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogs, fragments, and derivatives of such growth factors. Members of the transforming growth factor (TGF) supergene family, which are multifunctional regulatory proteins, are particularly preferred. Members of the TGF supergene family include the beta transforming growth factors (for example, TGF-β1, TGFβ2, TGF-β3); bone morphogenetic proteins (for example, BMP-1, BMP-2, BMP-3, BMP-4, BMP-5, BMP-6, BMP-7, BMP-8, BMP-9); heparin-binding growth factors (for example, fibroblast growth factor (FGF), epidermal growth factor (EGF), platelet-derived growth factor (PDGF), insulin-like growth factor (IGF)); Inhibins (for example, Inhibin A, Inhibin B); growth differentiating factors (for example, GDF-1); and Activins (for example, Activin A, Activin B, Activin AB).

Thus, as can be seen from the above, application of the foregoing biomaterial composition to the site of a bone defect, e.g., one resulting from injury, infection, malignancy or developmental malformation, leads to rapid new bone in-growth by one or more mechanisms such as osteogenesis, osteoconduction and osteoinduction.

In the foregoing description, the method and apparatus of the present invention have been described with reference to a specific example. It is to be understood and expected that variations in the principles of the method and apparatus herein disclosed may be made by one skilled in the art and it is intended that such modifications, changes, and substitutions are to be included within the scope of the present invention as set forth in the appended claims. The specification and the drawings are accordingly to be regarded in an illustrative rather than in a restrictive sense.

1. A putty form biomaterial composition, having improved flowable yet cohesive characteristics, the composition comprising:
a collagen scaffold, having an insoluble fibrous collagen and a soluble collagen formulation;
a biphasic ceramic granule, having a hydroxyapatite and beta-tri-calcium phosphate formulation; and
at least one structural polyhydroxy compound,
wherein addition of at least one hydrating agent gives the composition its putty form, but either individually or in some combination the at least one structural polyhydroxy compound maintains the flowable yet cohesive characteristic of the putty form.

2. The putty form biomaterial composition according to claim 1, wherein the at least one structural polyhydroxy compound comprises at least one of: a polysaccharide, a liquid polyhydroxy compound ester, a liquid solution of solid polyhydroxy compound, a liquid solution of solid polyhydroxy compound ester, a polyethylene glycol polymer, a poloxamer block co-polymer, an alginate and a chitosan.

3. The putty form biomaterial composition according to claim 2, wherein the liquid polyhydroxy compound and liquid polyhydroxy compound ester derivative include compounds which in the pure or highly concentrated state and at an ambient temperature are flowable liquids.

4. The putty form biomaterial composition according to claim 2, wherein the solid polyhydroxy compound and solid polyhydroxy compound ester derivative include compounds which in the pure or concentrated state and at an ambient temperature are normally solid or semi-solid but are soluble in a suitable solvent to provide a liquid composition.

5. The putty form biomaterial composition according to claim 2, wherein the polyhydroxy compounds possess from 2 to about 18 carbons and include such classes of compounds as the acyclic polyhydric alcohols, non-reducing sugars, sugar alcohols, sugar acids, monosaccharides, disaccharides, water-soluble or water dispersible oligosaccharides, polysaccharides and known derivatives of the foregoing.

6. The putty form biomaterial composition according to claim 2, wherein the polyhydroxy compound ester derivatives include liquid and solid monoesters and diesters of glycerol.

7. The putty form biomaterial composition according to claim 2, wherein the polyethylene glycol polymer is utilized when solubility at greatly reduced low temperatures is needed.

8. The putty form biomaterial composition according to claim 2, wherein the poloxamer block copolymer is utilized where a solubilizer and stablilizer that conforms to the NF 19 specifications is needed.

9. The putty form biomaterial composition according to claim 2, wherein the alginate is utilized where a thermally stable cold setting in the presence of ions is needed.

10. The putty form biomaterial composition according to claim 2, wherein the chitosan is utilized where a substance that is positively charged—and soluble in acidic to neutral solution is needed.

11. The putty form biomaterial composition according to claim 1, further comprising:
at least one biocompatible fluid lubricant.

12. The putty form biomaterial composition according to claim 1, further comprising:
at least one biologically active agent.

13. A biomaterial composition for implantation as a bone graft, the composition comprising:
a malleable, aqueous collagen;
a granular biphasic calcium phosphate dispersed within the collagen; and
at least one polyhydroxy compound contributing to improved flow and cohesive characteristics of the composition.

14. The biomaterial composition according to claim 13, wherein the at least one polyhydroxy compound comprises at least one of: a polysaccharide, a liquid polyhydroxy compound ester, a liquid solution of solid polyhydroxy compound, a liquid solution of solid polyhydroxy compound ester, a polyethylene glycol polymer, a poloxamer block co-polymer, an alginate and a chitosan.

15. The biomaterial composition according to claim 14, wherein the liquid polyhydroxy compound and liquid polyhydroxy compound ester derivative include compounds which in the pure or highly concentrated state and at an ambient temperature are flowable liquids.

16. The biomaterial composition according to claim 14, wherein the solid polyhydroxy compound and solid polyhydroxy compound ester derivative include compounds which in the pure or concentrated state and at an ambient temperature are normally solid or semi-solid but are soluble in a suitable solvent to provide a liquid composition.

17. The biomaterial composition according to claim 14, wherein the polyhydroxy compounds possess from 2 to about 18 carbons and include such classes of compounds as the acyclic polyhydric alcohols, non-reducing sugars, sugar alcohols, sugar acids, monosaccharides, disaccharides, water-soluble or water dispersible oligosaccharides, polysaccharides and known derivatives of the foregoing.

18. The biomaterial composition according to claim 14, wherein the polyhydroxy compound ester derivatives include liquid and solid monoesters and diesters of glycerol.

19. The biomaterial composition according to claim 14, wherein the polyethylene glycol polymer is utilized when solubility at greatly reduced low temperatures is needed.

20. The biomaterial composition according to claim 14, wherein the poloxamer block copolymer is utilized where a solubilizer and stablilizer that conforms to the NF 19 specifications is needed.

21. The biomaterial composition according to claim 14, wherein the alginate is utilized where a thermally stable cold setting in the presence of ions is needed.

22. The biomaterial composition according to claim 14, wherein the chitosan is utilized where a substance that is positively charged—and soluble in acidic to neutral solution is needed.

23. The biomaterial composition according to claim 13, further comprising:
at least one biocompatible fluid lubricant.

24. The biomaterial composition according to claim 13, further comprising:
at least one biologically active agent.
